(19) United States
(12) Patent Application Publication
(10) Pub. No.: US 2021/0113625 A1
(43) Pub. Date: Apr. 22, 2021

(54) EXTRACELLULAR VESICLES DERIVED FROM MESENCHYMAL STEM CELLS

(71) Applicants: HEALTH AND BIOTECH FRANCE (H & B France), Paris (FR); BEIJING HEALTH AND BIOTECH Co., Ltd., BEIJING (CN)

(72) Inventors: Angelita DE FRANCISCO, Paris (FR); ZhongChao HAN, BEIJING (CN)

(21) Appl. No.: 17/251,120

(22) PCT Filed: Jun. 11, 2019
(86) PCT No.: PCT/EP2019/065232

§ 371 Date: Dec. 10, 2020

(30) Foreign Application Priority Data
Jun. 11, 2018 (IB) PCT/IB2018/000796

(51) Int. Cl. A61K 35/28; C12N 5/0775

(52) U.S. Cl. CPC A61K 35/28; C12N 5/0665; C12N 2502/115; C12N 2501/2301; C12N 2501/2304; C12N 2501/165

(57) ABSTRACT

The present invention discloses a composition comprising extracellular vesicles (EVs) of placenta tissue derived CD106^{high} CD151⁺Nestin⁺ mesenchymal stem cells (MSCs). In a first aspect, the invention relates to a particular method to prepare these EVs. In a second aspect, the invention relates to a therapeutic, a diagnostic, a veterinary or a cosmetic composition comprising the extracellular vesicles (EVs) obtained by said particular method. In a third aspect, the invention relates to a composition comprising these EVs, for use as a medicament for treating subjects suffering from an ischemic disease, a disorder of the circulatory system, an immune disease, an organ injury or an organ function failure.

### BACKGROUND OF THE INVENTION

The therapeutic potential of mesenchymal stem cells (MSCs) for the repair and regeneration of damaged tissues has been widely studied both at the pre-clinical and clinical stages. MSCs mediate immuno-modulatory as well as pro-regenerative activities. For e.g., MSCs have been shown to improve wound healing in diabetic mice by promoting epithelialization, angiogenesis, and granulation tissue formation. Other studies have shown that bone marrow-derived MSCs are effective in treating wounds, including chronic wounds, by facilitating angiogenesis and scar reduction. The direct application of MSCs derived from the bone marrow or umbilical cord to patients with chronic non-healing wounds leads to wound closure and skin reconstruction.

In particular, PCT/EP2017/082316 presents a method to prepare CD106^{high} CD151⁺Nestin⁺ MSC for several therapeutic applications, such as for e.g., vascular diseases and wound. These MSC exhibit enhanced pro-angiogenic activities, among other activities.

The mechanisms underlying the healing properties of the MSCs have been studied. Interestingly, previous results from Shabbir & al obtained with wound healing assays did not allow to conclude that the MSCs themselves differentiate to replace the damaged tissues (fibroblasts). Instead, it has been shown that MSCs facilitate the migration of fibroblasts, an integral part of the healing process, even without direct contact (Shabbir & al 2015). This emphasizes the importance of paracrine signaling between the MSCs and the damaged cells.

This paracrine effect is thought to be the result of the secretion by the MSCs of various factors and of extracellular vesicles (EV). These vesicles comprise a lipid membrane bi-layer similar to that of the original MSCs, and carry various proteins, RNA messengers and miRNA (cargo) from this MSCs, factors that increase the endogenous mechanisms of repair and regeneration.

Extracellular vesicles (EVs), such as exosomes and microvesicles, have indeed been previously identified as major mediators of these paracrine effects of cells, acting as signaling mediators in immune biological processes (Raposo & al 1996). Since then, EVs have been found to mediate the interaction between various immune cell types and also between tumor and immune cells. Depending on the cell source, EVs can promote or suppress pro-inflammatory responses.

Cells, and MSCs in particular, can release a number of different vesicle types into their extracellular environment. Collectively named EVs, they include exosomes (30-150 nm), microvesicles that derive as bud offs from the plasma membrane (100-1,000 nm), and apoptotic bodies (>500 nm). They contain lipids, proteins and RNAs. They mediate targeted intercellular signaling in physiological and pathophysiological communication processes.

EVs isolated from MSCs have been gaining momentum as a novel strategy for accessing the therapeutic effects of stem cells without the risks and difficulties of administering the cells to patients. MSC-EVs provide indeed several key advantages over cellular products, which justify the efforts to translate EVs into the clinics:
MSC-EV can be safely administered.

MSC-EVs have been applied to an increasing amount of different animal models and have been tested in a patient suffering from steroid-refractory acute graft-versus-host disease (acute GvHD) as well as in a patient cohort with chronic kidney disease (Giebel & al 2017). So far, the MSC-EV administration appears to be safe in humans and all tested animal models.

In contrast to cellular products, the EVs cannot self-replicate and thus lack any endogenous tumor-formation potential.

It is known that biological features and functions of cells can be affected and re-programmed by environmental factors. Since EVs lack elaborated metabolic activities, it appears less likely that their function can be reprogrammed by the environment. Thus the biological activity and functional properties of EVs can be defined and controlled more precisely than for cells.

With average sizes below 200 nm, EVs can be sterilized by filtration. This massively reduces the risk of biological contamination of respective therapeutics.
EVs are much easier to handle than cells.

Freezing, thawing and storage conditions appear to be less critical for the EVs than for cells. For the bed-side preparation of cellular transplants the personal has to be specifically trained which might be dispensable for the bed-side preparation of EV-based therapeutics.

Therapeutic EVs might be produced from supernatants of cell lines, whose cells should not be used for cellular therapies themselves. Thus, EVs can be produced in a scaled manner much easier than cellular therapeutics.

The use of MSC-derived EVs for cell-free therapy is gaining momentum (Phinney & Pittenger—Stem Cells. 2017), and several studies have demonstrated the role of EV secreted by MSCs in angiogenesis:
exosomes secreted by MSCs promote endothelial cell angiogenesis by transferring miR-125a (Liang an al. J. Cell Sci. 2016),
exosomes released from human induced pluripotent stem cells-derived MSCs facilitate cutaneous wound healing by promoting collagen synthesis and angiogenesis (Zhang, J.& al. 20152015),
MSC exosomes induce proliferation and migration of normal and chronic wound fibroblasts, and enhance angiogenesis in vitro (Shabbir & al. 2015),
Human umbilical cord MSC exosomes enhance angiogenesis through the WNT4/catenin pathway (Zhang, B. & al. 2015).

A review by Than UTT & al of 2017 reports other studies having indeed shown that EVs are involved in the control of a number of cellular processes necessary for wound healing. In particular, EVs could influence coagulation, cell proliferation, migration, angiogenesis, collagen production and remodeling of the extracellular matrix. In addition to carrying information from the original secretory cells, EV mRNAs and miRNAs can also promote biological processes, including proliferation, angiogenesis, and apoptosis.

EVs regulation of cell proliferation, an essential process for wound healing, has been shown with EVs derived from a multitude of cell types, such as MSCs, fibroblasts, murine embryonic stem cells, and human endothelial pro-progenitors. In particular, internalization of MSC-EV by fibroblasts produces a dose-dependent increase in the proliferation and migration of these fibroblasts, whether from normal donors or patients with chronic wounds.

EVs can promote cell proliferation by activating not only signaling pathways directly involved in cell cycle stimulation, but also signaling pathways involved in regulating growth factor expression. In addition, this overexpression of growth factors can act as paracrine or autocrine signals to stimulate the cell proliferation in turn.

The migration of endothelial cells, crucial for vascular repair and regeneration, has also been shown to be influenced by EVs released from cells such as keratinocytes or human CSMs.

All types of EV (microvesicles and exosomes) contribute to varying degrees to the regulation of vessel formation by increasing the expression of pro-angiogenic factors. For example, exosomes released by human embryonic MSCs and human endothelial cells enhance angiogenesis by promoting proliferation and migration of endothelial cells to the wound site. A larger number of blood vessels, compared to the control treatment, was observed at sites treated with exosomes.

All of these assays suggest that MSC-EVs can be safely used in the treatment of wounds and in regenerative vascular medicine.

Dongdong Ti & al (Journal of translational medicine, 2015) described extra-cellular vesicles purified from umbilical cord MSCs that are pre-conditioned with the pro-inflammatory factor LPS.

Yunbing Wu & al (BioMed Research International, 2017) described anti-inflammatory properties of extra-cellular vesicles derived from unstimulated mesenchymal stem cells obtained from human umbilical cord.

None of these two publications proposes to add IL1β and/or IL4 for stimulating the MSCs in order to obtain EVs that are enriched in the CD106 angiogenic marker.

More generally, none of the prior art documents ever proposed to treat MSCs with IL4, let alone to improve their pro-angiogenic properties. In this context, the present inventors have shown in WO 2018/108859 that cultivating MSCs with IL4 and ILβ lead to a significant and surprising increase of the surface level of pro-angiogenic surface markers such as CD106. In particular, as shown in example 4 of WO 2018/108859, the increase of CD106 expression observed with a combination of IL1β and IL4 is 3 folds higher than the increase observed with IL1β alone. And the increase of CD106 expression observed with a combination of IL1β and IL4 is 5 folds higher than the increase observed with IL4 alone. This had never been observed before.

The present work herein demonstrates that the pro-angiogenic and anti-inflammatory activities of CD106^{high} CD151⁺Nestin⁺ MSCs produced according to the method described in PCT/EP2017/082316 (WO 2018/108859) are substantially conveyed by the EVs derived from these MSCs. The inventors therefore propose to use a biological product containing these EVs, because—as their producing MSCs—they express enhanced level of pro-angiogenic/pro-inflammatory surface proteins. Accordingly, this biological product exhibits the same enhanced pro-angiogenic activity as the MSCs of origin, without their constraints of use in clinical and industrial practice.

In addition to their therapeutic potential, EVs can be used as biomarkers, in particular in the diagnosis of cancer. Among the 35 clinical trials currently underway relating cancer to exosomes, approximately two-thirds relate to diagnostics, and the rest to therapeutics (Roya & al 2018).

### DETAILED DESCRIPTION OF THE INVENTION

In a first aspect, the present invention thus relates to a composition comprising extracellular vesicles (EVs) of CD106^{high} CD151⁺Nestin⁺ MSCs that have been produced according to the method described in PCT/EP2017/082316 (published as WO 2018/108859).

This producing method comprises the two following general steps:
(i) Culturing mesenchymal stem cells obtained from a biological tissue or fluid in a first culture medium deprived of growth factors, so as to generate a population of cultured undifferentiated mesenchymal stem cells,
and
(ii) contacting said population of cultured undifferentiated mesenchymal stem cells with a 20 second culture medium containing pro-inflammatory growth factors or inflammatory mediators, thereby generating the CD106^{high} CD151⁺Nestin⁺ mesenchymal stem cells of interest that will be used to produce the EVs of the invention.

Said “population of undifferentiated MSCs” can be obtained by collecting the mononuclear cells present in a biological tissue or fluid and growing them in a first culture medium. These mononuclear cells can be obtained by any conventional means, e.g., by enzymatic digestion or explant culture of perinatal tissue pieces (Otte et al, 2013) or isolation from biological fluids (Van Pham et al, 2016).

Explant culture is a particularly preferred process for deriving such MSCs from umbilical cords, as exposed in example 3 below.

Typically, this process requires to remove the sample from the transport solution, to cut it in sections (roughly 2-3 cm long), to disinfect them with antibiotics and antifungal agents that are rinsed afterwards, to recover the tissue and dispose pieces of said tissue in flasks for them to adhere (preferably without medium, at room temperature), before complete medium is added carefully on the adhered explants and keep incubated at 37° C. for several days. The migrated cells are eventually collected with appropriate tools and maintained in culture in the appropriate first medium (see below) until they reach the target confluency.

Said “first culture medium” can be any classical medium commonly used to favor growth of living primary cells. Preferably, it does not contain any growth factors nor any differentiation factors.

The skilled person well knows what kind of culture media can be used as “first culture medium”. They are for example DMEM, DMEM/F12, MEM, alpha-MEM (α-MEM), IMDM, or RPMI. Preferably, said first culture medium is DMEM (Dulbecco's Modified Eagle's Medium) or DMEM/F12 (Dulbecco's Modified Eagle's Medium: Nutrient Mixture F-12).

More preferably, said first culture medium contains 2-20% or 2-10% of fetal bovine serum. Alternatively, said first culture medium may contain 1-5% platelet lysate. A most preferred medium contains 2-20% or 2-10% of fetal bovine serum and 1-5% platelet lysate.

It is also possible to use as first culture medium a medium which is devoid of serum or platelet lysate, provided that it contains other appropriate agents favoring the growth of primary living cells.

In a preferred embodiment, said “biological tissue” is any portion of a placental tissue, or of umbilical cord. In particular, it can include or consist in placental cotyledons, the amnion membrane or the chorionic membrane of the placenta. Also, it can be the Wharton jelly found in the umbilical cord. It can include the veins and/or the arteries, or be deprived thereof.

In another embodiment, said “biological fluid” is a sample of umbilical cord blood, of placenta blood or of amniotic fluid, which have been harmlessly collected from a woman or a mammal in general. For example, these tissues and fluids can be obtained after the delivery of a baby or an offspring, without any invasive proceedings.

Said population of undifferentiated MSCs is preferentially a population of mesenchymal stem cells seeded on a plastic surface, which has been cultured in said first culture medium devoid of any growth factor until the cells reach a confluency of 85-90%.

Regularly, the cells are phenotypically characterized by FACS or any conventional means, in order to detect the level of the surface markers CD73, CD90, CD105, CD166, CD45, CD34 and HLA-DR.

When 95% of the cells express the positive surface markers CD73, CD90, CD105 and CD166, and less than 2% express the negative surface markers CD45, CD34 and HLA-DR, the cells are trypsinized and seeded again at a lower density, e.g. at a density of 1000 to 5000 MSCs per cm² into a second culture medium.

Preferably, said “second culture medium” is any classical medium commonly used to favor living primary cells growth. It can be the same medium as the “first culture medium”, or it can be another one, chosen for example among DMEM, DMEM/F12, MEM, alpha-MEM (α-MEM), IMDM, or RPMI. More preferably, said second culture medium is DMEM (Dulbecco's Modified Eagle's Medium) or DMEM/F12 (Dulbecco's Modified Eagle's Medium: Nutrient Mixture F-12).

Even more preferably, said second culture medium contains serum or platelet lysate, for example between 2-20% of fetal bovine serum and/or 1-5% platelet lysate. A most preferred second medium is DMEM containing 2-20% of fetal bovine serum and 1-5% platelet lysate. It is also possible to use as second culture medium a medium which is devoid of serum or platelet lysate, provided that it contains other appropriate agents favoring the growth of primary living cells.

When the cells reach 40-50% confluency, pro-inflammatory growth factors or inflammatory mediators are added to the second culture medium and the cells are cultured in said medium until they reach 90-95% confluency.

Said “pro-inflammatory growth factors” are typically interleukins or chemokines that are known to have a pro-inflammatory effect. Examples of interleukins that can be added in the second culture medium include TNFα, IL1, IL4, IL12, IL18, and IFNγ. Examples of chemokines that can be added in the second culture medium include CXCL8, CXCL10, CXCL1, CXCL2, CXCL3, CCL2, and CCLS. Other inflammatory mediators (such as anti-inflammatory agents) can be used.

In a preferred embodiment, at least two pro-inflammatory growth factors are added in the second culture medium defined above. These at least two pro-inflammatory growth factors are chosen in the group consisting of: TNFα, IL1, IL4, IL12, IL18, and IFNγ. In a more preferred embodiment, said pro-inflammatory growth factors are chosen among IL1, IL4, IL12, IL18. Even more preferably, they are IL1 and IL4.

A typical concentration of growth factor(s) that can be added to the MSCs is comprised between 1-200 ng/mL, preferably between 1-100 ng/mL, more preferably between 10-80 ng/mL. Preferably, the culturing step of the MSCs with the growth factor(s) lasts for at least one day, more preferably for two days.

The term “IL1” herein designates any isoform of Interleukin 1, in particular, IL1α and IL1β. IL1 isoforms may be of various origins, depending on the intended application. For example, animal IL1 may be used for veterinary applications. Preferably, only IL1β is added in the second culture medium of the invention. In this particular embodiment, the concentration of added Interleukin 1β can be comprised between 1-100 ng/mL, preferably between 1-50 ng/mL, more preferably between 10-40 ng/mL.

Human IL1beta (IL1β or IL1b) is referenced to as accession number NP_000567.1. Recombinant protein is commercially available in GMP conditions (RnD systems, Thermofisher, Cellgenix, Peprotech).

The term “IL4” herein designates any isoform of Interleukin 4. IL4 may be of various origins, depending on the intended application. For example, animal IL4 may be used for veterinary applications.

Human IL4 is referenced to as accession number AAA59149. Recombinant protein is commercially available in GMP conditions (RnD systems, Thermofisher, Cellgenix, Peprotech).

Any mixture of different pro-inflammatory growth factors can be used in the said second medium. In particular, it is a preferred embodiment to use a mixture of IL1 and IL4, more precisely, of IL1β and IL4, as disclosed in the experimental part below.

In this particular embodiment, the added Interleukin 1β has a concentration comprised between 1-100 ng/mL, preferably between 1-50 ng/mL, more preferably between 10-40 ng/mL. and the added IL4 has a concentration comprised between 1-100 ng/mL, preferably between 1-50 ng/mL, more preferably between 10-40 ng/mL in the second culture medium. Preferably, the culturing step with Interleukin 1β and IL4 lasts for at least one day, more preferably for two days. Typically, the concentration of added interleukin 1δ is of 10 ng/mL, and the concentration of added interleukin IL4 is of 10 ng/mL. The cells are therefore preferably cultivated in a culture medium containing 10 ng/mL of both Interleukin 1β and IL4, this culture step lasting for example two days.

The cells can be phenotypically characterized by any conventional means, in order to detect the level of surface markers CD73, CD90, CD105, CD166, CD45, CD34 and HLA-DR during their production. These markers are well-known in the art. Antibodies useful for detecting the expression level of these markers are all commercially available.

Expression of these cell surface markers may be notably assessed using well known technologies such as cell membrane staining using biotinylation or other equivalent techniques followed by immunoprecipitation with specific antibodies, flow cytometry, western blot, ELISA or ELISPOT, antibodies microarrays, or tissue microarrays coupled to immunohistochemistry. Other suitable techniques include FRET or BRET, single cell microscopic or histochemistry methods using single or multiple excitation wavelength and applying any of the adapted optical methods, such as electrochemical methods (voltametry and amperometry techniques), atomic force microscopy, and radio frequency methods, e.g. multipolar resonance spectroscopy, confocal and non-confocal, detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), magnetic resonance imaging, analysis by polyacrylamide gel electrophoresis (SDS-PAGE); HPLC fractionation, MALDI-TOF Mass Spectroscopy; Liquid Chromatography/Mass Spectrometry/Mass Spectrometry (LC-MS/MS). Preferably, the levels of cell surface markers are assessed by FACS.

Specifically, producing the MSCs of interest typically requires to:

a) collect the mononuclear cells contained in a perinatal biological tissue or fluid,

b) allow said mononuclear cells to grow into a first culture medium until they reach 85-90% confluency, preferably on a plastic surface,

c) once 95% of the cells express the positive markers CD73, CD90, CD105 and CD166, and less than 2% express the negative markers CD45, CD34 and HLA-DR, seed the cells at a density of 1000 to 5000 MSCs per cm² into a second culture medium,

d) add between 1-100 ng/mL of inflammatory mediators or pro-inflammatory growth factors once the cells reach 40-50% confluency,

e) collect the cells when they reach 90-95% confluency.

The collected cells may then be phenotypically characterized by FACS or any conventional means, in order to detect the level of surface markers CD73, CD90, CD105, CD166, CD45, CD34 and HLA-DR. Said first and second culture media have been described above.

In step d) of said method, the typical concentration of added growth factor(s) is comprised between 1-200 ng/mL, preferably between 1-100 ng/mL, more preferably between 10-80 ng/mL. Preferably, the culturing step with growth factor(s) lasts for at least one day, more preferably for two days.

In step d) of said method, the concentration of added Interleukin 1β or IL4 can be comprised between 1-100 ng/mL, preferably between 1-50 ng/mL, more preferably between 10-40 ng/mL. Preferably, the culturing step with Interleukin 1β and IL4 lasts for at least one day, more preferably for two days. Typically, the concentration of added interleukin 1β is of 10 ng/mL, and the concentration of added interleukin IL4 is of 10 ng/mL. The cells are therefore preferably cultivated in a culture medium containing 10 ng/mL of both Interleukin 1β and IL4, this culture step lasting for example two days.

The final collected cells will be the “MSCs of the invention”, “cell culture of interest”, or “CD106^{high} CD151⁺Nestin⁺ MSCs of interest” or the “EVs producing cells”. This cell culture typically comprises over 60%, preferably between 60 and 70%, preferably over 70%, preferably over 80%, more preferably over 90% and even more preferably over 95% of cells expressing CD106. Moreover, it comprises over 98%, preferably over 99% of cells expressing CD151. Moreover, it comprises over 98%, preferably over 99% of cells expressing Nestin, Finally, it comprises over 95%, preferably over 96%, preferably over 97%, preferably over 98% of cells expressing the positive markers CD73, CD90, CD105 and CD166, and comprises less than 2% cells expressing the negative markers CD45, CD34 and HLA-DR.

CD106 (also known as VCAM-1 for “vascular cell adhesion protein 1”) is known to have three isoforms. NP_001069.1, NP_542413.1 and NP_001186763.1 are the sequences of the isoforms a, b and c respectively. Antibodies to detect the level of expression of this particular biomarker are commercially available (for example by Thermofisher, Abcam, OriGen, etc.). The expression of this marker at the surface of the MSCs is very important, as it triggers pro-angiogenic activities that are essential for their therapeutic use.

The nestin biomarker is referenced under the number NP_006608.1 in humans. Antibodies to detect the level of expression of this particular biomarker are commercially available (for example by Thermofisher, Abcam, etc.).

The CD151 biomarker is referenced under the number NP_620599 in humans. Antibodies to detect the level of expression of this particular biomarker are commercially available (for example by Invitrogen, Sigma-Aldrich, Abcam, etc.).

More specifically, producing the MSCs of interest typically requires to:
a) Optionally separately collecting placental tissues from multiple donors;
b) Optionally washing the placental tissue three times using 1×PBS, dissected in 1 mm³ cubes and washing the cubes tissue again to remove most of the blood from the tissue.
c) Optionally digesting the placental tissue of each donor separately with collagenase, centrifuging the digested tissue and collecting the mononuclear cells,
d) Seeding the collected mononuclear cells into a culture medium;
e) Trypsinizing and passage the cells once they reach 85-90% confluence;
f) Characterizing the cells based on the percentage of cells which express positive markers CD73, CD90, CD105 and CD166, and negative markers CD45, CD34 and HLA-DR;
g) Seeding the cells in a culture medium containing 90% Dulbecco's Modified Eagle's Medium/F12-Knockout (DMEM/F12-KO) and 10% FBS and growth factors at a seeding density of 1000 to 5000 MSCs per cm² when they comprise at least 95% of the positive markers and at most 2% of the negative markers,
h) Adding between 1-100 ng/mL of interleukin 1β and optionally between 1-100 ng/mL of IL4 when they are 40-50% confluent;
i) Trypsinizing and collecting the cells once they reach 90-95% confluence; and
j) Optionally characterizing the cells based on the percentage of cells which express positive markers CD73, CD90, CD105 and CD166, and negative markers CD45, CD34 and HLA-DR.

The cells obtained by means of these methods are then used to produce the EVs of the invention.

Extracellular vesicles (EVs), is the general term to designate cell vesicles ranging approximately 30 nm to few μm in size. Among them, exosomes comprise the most prominently described classes of EV. Exosomes have a diameter lower than about 150 nm and are derivatives of the endosomal compartment. EV contain cytosolic and membrane proteins derived from the parental cells. The protein content of EV depends on their cellular origin and EV are enriched for certain molecules, especially endosome-associated proteins (e.g. CD63) and proteins involved in multivesicular bodies formation, but also contain targeting/adhesion molecules. Remarkably, EV contain not only proteins but also functional mRNAs, long non-coding RNAs and miRNAs, and in some cases, they have been shown to deliver these genetic materials to recipient cells.

By “extracellular vesicles” or “EVs”, it is meant membranous vesicles released by cells in their microenvironment from their plasma membrane, or intracellular vesicles retrieved after cell membrane lysis. In the context of the present invention, EVs typically have a diameter lower or equal to about 500 nm, in particular between about 30 and about 500 nm, or between about 40 and about 500 nm, or between about 50 and about 250 nm. EVs are surrounded by a phospholipid membrane, which preferably contains relatively high levels of cholesterol, sphingomyelin, and ceramide and preferably also contains detergent-resistant membrane domains (lipid rafts).

The membrane proteins of the EVs have the same composition as the cell membranes. EVs are generally characterized by the presence of Actin 6, proteins involved in membrane transport and fusion (such as Rab, GTPases, annexins, and flotillin), components of the endosomal sorting complex required for transport (ESCRT) complex (such as Alix), tumor susceptibility gene 101 (TSG101), heat shock proteins (HSPs, such as HSPA8, HSP90AA1, HSC70 and HSC90), integrins (such as CD62L, CD62E or CD62P), and tetraspanins (in particular CD63, CD81, CD82, CD53, CD9, and/or CD37). In the examples below, they are identified by the combination of the markers CD9 and CD81, and by the absence of calnexin.

It is also within the scope of the invention to consider using the secretome of the CD106^{high CD}151⁺Nestin⁺ MSCs that have been produced according to the method described in PCT/EP2017/082316. The term “secretome” herein designates all the factors that are secreted by a cell, including the EVs, the proteins (growth factors, chemokines, cytokines, adhesion molecules, proteases, etc.), lipids, micro-RNAs, mRNAs. The secretome of the CD106^{high} CD151⁺Nestin⁺ MSCs that have been produced according to the method described in PCT/EP2017/082316 is thought to share the same pro-angiogenic biological effects as the CD106^{high} CD151⁺Nestin⁺ MSCs described in PCT/EP2017/082316.

EVs may be purified from the MSCs cells of interest by various methods, such as methods described in Konoshencko & al 2018 or in Lai & al 2010.

Differential Centrifugation:

The method consists of several steps including at least the following three steps 1) to 3):

1) a low speed centrifugation to remove cells and cellular debris (<10,000×g),

2) a higher speed spin to eliminate larger vesicles, and finally:

3) a high speed centrifugation to pellet the EV (>100,000×g).

The obtained EV preparation is further purified and the isolated vesicles are selected according to their size by microfiltration of suspension.

Density Gradient Centrifugation:

This approach combines ultracentrifugation with sucrose density gradient. More specifically, density gradient centrifugation is used to separate EV from non-vesicular particles, such as proteins and protein/RNA aggregates. Thus, this method separates vesicles from the particles of different densities. The adequate centrifugation time is very important, otherwise contaminating particles may be still found in EV fractions if they possess similar densities. Recent studies suggest application of the EV pellet from ultracentrifugation to the sucrose gradient before performing centrifugation.

Size Exclusion Chromatography:

Size-exclusion chromatography is used to separate macromolecules on the basis of size and shape not molecular weight. The technique applies a column packed with porous polymeric beads containing multiple pores and tunnels. The molecules pass through the beads depending on their diameter. It takes longer time for molecules with small radii to migrate through pores of the column, while macromolecules elute earlier from the column. Size-exclusion chromatography allows precise separation of large and small molecules. Moreover, different eluting solutions can be applied to this method.

Ultrafiltration:

Ultrafiltration membranes can also be used for isolation of EV. Depending on the size of microvesicles, this method allows the separation of EV from proteins and other high molecular weight macromolecules. EV may also be isolated by trapping them via a porous structure. Most common filtration membranes have pore sizes of 0.8 μm, 0.45 μm or 0.22 μm and can be used to collect EV larger than 800 nm, 400 nm or 200 nm. In particular, a micropillar porous silicon ciliated structure was designed to isolate 40-100 nm EV. During the initial step, the larger vesicles are removed. In the following step, the EV population is concentrated on the filtration membrane. The isolation step is relatively short, but the method requires pre-incubation of the silicon structure with PBS buffer. In the following step, the EV population is concentrated on the filtration membrane.

Polymer-Based Precipitation:

Polymer-based precipitation technique usually includes mixing the biological fluid with polymer-containing precipitation solution, incubation at 4° C. and ultracentrifugation. One of the most common polymers used for polymer-based precipitation is polyethylene glycol (PEG), preferably PEG 6000 or PEG 8000. The precipitation with this polymer has a number of advantages, including mild effects on isolated EV and usage of neutral pH. Several commercial kits applying PEG for isolation of EV were generated. The most commonly used kit is ExoQuick™ (System Biosciences, Mountain View, Calif., USA). Recent studies demonstrated that the highest yield of EV was obtained using ultracentrifugation with ExoQuick™ method.

Immunological Separation:

Several techniques of immunological separation of EV have been developed, based on surface EV extrinsic or intrinsic membrane associated proteins or EV intracellular proteins. These methods are however generally applied mainly for detection, analysis and quantification of EV proteins.

In the examples below, ultrafiltration has been used.

The EVs obtained by purifying the MSCs cells obtained from the above-mentioned producing method are hereafter referred to as the “EVs of the invention”. They display an enhanced pro-angiogenic activity (as their producing MSCs cells), as compared with the EVs of the prior art.

As shown in example 8 below, these EVs are in particular characterized in that they express:
(i) CD106 at a detectable level, and
(ii) CD200 at a detectable level.

Importantly, they express the pro-angiogenic markers CD106/VCAM and CD200 at higher level than the EVs of the prior art (see FIG. 3). They also express a number of other angiogenic markers, such as FGF7, CCL2 and Angiopoietin 1 (see FIG. 4).

These markers are well-known in the art and antibodies detecting same are commercially available. Their presence can be assessed by any conventional means, such as western blot.

According to the present invention, a EV “expresses a marker at a detectable lever” if said marker is present at a significant level, i.e., if the signal associated to the staining of said marker (typically obtained with an antibody recognizing said marker, said antibody being for example coupled to a fluorescent dye) which is measured for said EV is superior to the signal corresponding to the staining of EVs being known as not expressing said marker. The skilled person is well aware of how to identify said cells/markers so that these protocols do not need to be detailed here.

The composition of the invention essentially comprises the extracellular vesicles (EVs) of the invention produced by the MSC cells disclosed in PCT/EP2017/082316.

In the context of the present invention, the number of EVs present in a composition is preferably determined using a NanoSight apparatus (commercialized by Malvern), in which case the number of EV is referred to as “pp”, corresponding to the number of particles detected by NanoSight apparatus.

Typically, the composition of the invention contains between 1×10⁸ and 1×10¹⁴ ppEV per mL, more preferably between 1×10¹¹ and 1×10¹² ppEV per mL.

In a second aspect, the present invention also relates to a method for preparing a composition comprising EVs of the MSCs obtained by the above-mentioned method, comprising:
a) Culturing said MSCs in a EV-free culture medium, under conditions permitting their expansion; and
b) Purifying the EVs from these cells.

“EV-free culture medium” that can be herein used are for example uncomplemented classic basal medium (such as alpha-MEM, DMEM, DMEM/F12 . . . ) or a classic basal medium supplemented with between 1 and 10%, preferably 5% more preferably 8% of vesicle-free platelet lysate. It does not contain any exogenous EV before being put in contact with the MSCs of the invention (once in contact with the MSCs of the invention, this medium begins to contain the EVs that are produced by the MSCs of the invention). Therefore, it does not contain any serum nor platelet lysate that may contain exogenous vesicles.

Said EV-free culture medium is preferably supplemented with added growth factor(s), whose concentration is comprised between 1-200 ng/mL, preferably between 1-100 ng/mL, more preferably between 10-80 ng/mL. More preferably, said culture medium is supplemented with Interleukin 1β and/or IL4 whose concentration is comprised between 1-100 ng/mL, preferably between 1-50 ng/mL, more preferably between 10-40 ng/mL. Typically, the concentration of added interleukin 1β in said medium is of 10 ng/mL, and the concentration of added interleukin IL4 in said medium is of 10 ng/mL. The cells are therefore preferably cultivated in a EV-free culture medium containing 10 ng/mL of both Interleukin 1β and IL4, this culture step lasting for example two days.

Conditions permitting expansion of the MSC cells have been described above. The important point is that cells should be in good condition, since cell death and apoptotic bodies could lead to contamination of the EV preparation. Conditions permitting amplification and maintenance of the MSC cells in exponential growth should thus be used, and EV should be purified before the end of the exponential phase of growth, i.e. before the plateau, when cell death becomes significant. For media containing animal driven components (e.g. serum), EV depletion of the medium should be conducted. This may be performed by spinning the culture medium at 100 000 g for 8-16 hours (for instance, overnight) at about 4° C.

The culture of the MSCs in said conditions typically lasts 1 to 7 days, preferably 1 to 5 days, more preferably 2 to 3 days, even more preferably for 72 h.

For therapeutic purposes, the whole method should preferably be performed under sterile conditions.

Purifying the EVs can be done by any of the above-mentioned processes (preferably by ultrafiltration as exposed in the experimental part below).

The results disclosed below also show that the methods of the invention enable to generate EVs expressing high level of the CD106/VCAM1 membrane protein. Importantly, this protein is associated to the expression of pro-angiogenic cytokines and pro-inflammatory proteins (Han Z. C., et al, Bio-medical Materials and Engineering 2017 & Du W. et al, Stem Cell research & therapy 2016. Therefore, the EVs of the invention, that express high level of the CD106 protein, can be used for their pro-angiogenic/pro-inflammatory efficiency.

Katoh & Katoh (Stem Cell Investig. 2019; 6: 10) mention that CD200 is involved in a variety of physiological and pathological processes at the crossroads of vascular remodeling and immune regulation. CD200 is a transmembrane protein that is expressed on a variety of cells, such as B and T lymphocytes, endothelial cells, neurons and pancreatic islet cells, and whose expression is upregulated by IL4. CD200 transduces signals through CD200R, a transmembrane protein. CD200-CD200R signaling plays a critical role in cancers and noncancerous diseases through the regulation of immunity and angiogenesis. For example, compared with CD200− B16 melanoma cells, CD200+ B16 melanoma cells exhibit enhanced tumorigenesis owing to the expansion of myeloid-lineage cells and increased tumor angiogenesis in Cd200r knockout mice.

Consequently, in a third aspect, the present invention relates to a composition comprising extracellular vesicles derived of the CD106^{high} CD151⁺Nestin⁺ MSCs produced according to the method described in PCT/EP2017/082316, for use for treating subjects suffering from an ischemic disease, a disorder of the circulatory system, an immune disease, an organ injury or an organ function failure. In other words, the invention relates to the use of said EVs for the manufacture of a medicament intended to be used for treating subjects suffering from an ischemic disease or from a disorder of the circulatory system. The medicament of the invention can also be applied to skin vascular capillary network and may include dermatological and cosmetic applications.

Preferably, the composition of the invention does not contain any cells; in particular, it does not contain any MSCs cells. It typically contains, as only active principle, only the EVs of the invention. It can also contain a pharmaceutical carrier or an adjuvant, as explained below.

The composition comprising the EVs of the invention is administered in therapeutically efficient amounts.

As used herein, a “therapeutically efficient amount” refers to an amount sufficient for the intended use. For the pro-angiogenic compositions of the invention, it refers to an amount sufficient to induce endothelial migration and/or proliferation.

The administered dose may vary depending on the subject age, body surface area or body weight, or on the administration route and associated bioavailability. Such dose adaptation is well known to those skilled in the art.

Any mammal may be treated with the compositions/EVs of the invention. Said mammal can be a pet (a dog, a cat, a horse, etc.) or a cattle animal (a sheep, a goat, a cow, etc.). It is obvious for the skilled person that, when an animal is to be treated according to the method of the invention, the initial undifferentiated MSCs will be obtained from a biological sample from the same animal species (allogenic graft) or from a similar species (heterologous graft), and the growth factors that are used in the second culture medium will correspond to those of the same animal species. For example, if a cat is to be treated, then the initial MSCs will be obtained from a perinatal tissue or biological fluid of a cat, and a cat IL1β (recombinant or not) will be added in the second culture medium, optionally along with cat IL4.

In a preferred embodiment, said mammal is a human being. In this case, the initial MSCs will be obtained from a perinatal tissue or from a biological fluid obtained from a woman, and human IL1β (recombinant or not) will be added to the second culture medium, optionally along with human IL4.

In this aim, the composition of the invention may be administered or topically applied to said subject by any conventional means. In this case, the present invention is drawn to a method for treating a subject suffering from an ischemic disease, a disorder of the circulatory system, an immune disease, an organ injury or an organ function failure, said method comprising the step of administering the composition described above to said subject. This administration may be performed by using an implanted reservoir or by injecting the EVs in situ in the muscle, or via intravenous injections or by any appropriate delivery system. The application may also be performed topically, by directly contacting the EVs with skin or a mucous membrane, or by applying the EVs with a device on the skin or on any mucous membrane, or by delivering the EVs by any appropriate delivery system to the skin or mucous membrane.

Preferably, said disease or disorder is chosen in the group consisting of: type-1 diabetes mellitus, type-II diabetes, GVHD, aplastic anemia, multiple sclerosis, Duchenne muscular dystrophy, rheumatoid arthritis, cerebral stroke, idiopathic pulmonary fibrosis, dilated cardiomyopathy, osteoarthritis, cirrhosis, liver failure, kidney failure, peripheral arterial occlusive disease, critical limb ischemia, peripheral vascular disease, heart failure, diabetic ulcer or any degenerative disease, synechia, endometrial disorder or fibrotic disorder of the gastro-intestinal tract such as anal fistula. More preferably, said disease or disorder is a peripheral arterial occlusive disease, a critical limb ischemia, a peripheral vascular disease, or a diabetic ulcer. In a particular embodiment, said disease or disorder is a skin or a mucous membrane disease, including (but not limited to) a diabetic ulcer, an ulcer, a trauma, a burn, a scald, a wound or a wound healing problem, Decubitus ulcer, a wart, etc.

The EVs of the invention may more precisely be used in a dermatological preparation whose aim is to treat skin pathologies such as burns, wounds, ulcers, scars, warts, or other diseases such as synechia or fibrotic disorders of the gastro-intestinal tract (for example anal fistula).

In another particular embodiment, said disease or disorder is anal fistula or endometrial injury.

Other applications are encompassed within the present application. In particular, it is possible to use the EVs and the compositions of the invention for diagnostic, dermatologic or cosmetic purposes, for example for regenerating the cells of the skin or of a mucosal membrane, improving the aspect of the skin or of a mucosal membrane, correcting a defect of the skin or of a mucosal membrane or for healing burning area of the skin or of the mucosal membrane.

In order to enhance the efficiency and facilitate the administration of the medicament of the invention, the EVs of the invention may be mixed with any agent, composition of agents or other biologically compatible material or device. The EVs of the invention may also be encapsulated or included in any appropriate delivery system or biocompatible material. The EVs or compositions containing same may be applied with a medical device, such as an endoscope, a stent, or a syringe, for example. It can be also applied topically by contacting the EVs with the skin or a mucosa.

For intravenous, intratumoral or intranasal administration, aqueous suspensions, isotonic saline solutions, or sterile, injectable solutions that contain pharmacologically compatible dispersing agents and/or wetting agents may be used. As an excipient, water, alcohols, polyols, glycerol, vegetable oils, etc., may be used.

For topical administration, compositions may be presented in the form of a gel, a paste, an ointment, a cream, a lotion, an aqueous or aqueous-alcohol liquid suspension, an oily solution, a dispersion of the lotion or serum type, an anhydrous or lipophilic gel, an emulsion with a liquid or semi-solid milk-type consistency obtained by dispersing a fatty phase in an aqueous phase or vice versa, suspensions or emulsions of a soft or semi-solid cream- or gel-type consistency, or alternatively microemulsions, microcapsules, microparticles, or vesicular dispersions of the ionic and/or nonionic type. These compositions are prepared according to standard methods. Moreover, a surfactant can be included in the composition in order to enable deeper penetration of EV. An agent enabling an increased penetration may be selected, for example, from mineral oil, ethanol, triacetin, glycerin and propylene glycol; cohesion agents are selected, for example, from the group comprising polyisobutylene, polyvinyl acetate, polyvinyl alcohol, and thickening agents.

Suitable unit dose administration formulations for oral administration notably include tablets, coated tablets, pills, capsules and soft gelatin capsules, oral powders, granules, solutions and suspensions.

When a solid composition in tablet form is prepared, the principal active ingredient may be mixed with a pharmaceutical vehicle, such as gelatin, starch, lactose, stearic acid or magnesium stearate, talc, gum arabic or analogues. The tablets may be coated with saccharose or other suitable materials or even be treated so as to have a prolonged or delayed activity and to release continuously a predetermined quantity of the active ingredient.

A capsule preparation may be obtained by mixing the active ingredient with a thinner and pouring the mixture obtained into soft or hard capsules, with excipients such as vegetable oils, waxes, fats, semi-solid or liquid polyols, etc.

A preparation in syrup or elixir form can contain the active ingredient together with a sweetener, an antiseptic, as well as an agent giving taste and a suitable dye. Excipients may be used, such as water, polyols, saccharose, invert sugar, glucose, etc.

Powders or water-dispersible granules may contain the active ingredient in a mixture with dispersing agents, wetting agents, and suspending agents, together with taste correctors and sweeteners.

For subcutaneous administration, any suitable pharmaceutically acceptable vehicle may be used. In particular, a pharmaceutically acceptable oil vehicle, such as sesame oil, may be used.

The present invention also targets a medical device containing the EVs of the invention. By “medical device”, it is herein encompassed any instrument, apparatus, implement, machine, appliance, implant, reagent for administering a therapeutic composition. In the context of the invention, said medical device is, for example, a patch, a stent, an endoscope, or a syringe.

The present invention also targets a delivery system containing the EVs of the invention. By “delivery system”, it is herein encompassed any system (medium or carrier) for administering a pharmaceutical product to a patient. It can be an oral delivery or a controlled-release system. In the context of the invention, said delivery system is for example liposomes, proliposomes, microspheres, micro- or nano-vesicles of biopolymers, lipids or nanoparticles.

In a preferred embodiment of the invention, the EVs of the invention are included in an hydrogel or another biocompatible material or excipient. Said hydrogel may include notably alginate sodium hydrogel, hyaluronic acid hydrogel, chitosan hydrogel, collagen hydrogel, HPMC Hydrogel, Poly-L-lysine hydrogel, Poly-L-glutamic acid hydrogel, polyvinyl alcohol (PVA) hydrogel, polyacrylic acid hydrogel, polymethylacrylic acid hydrogel, polyacrylamide (PAM) hydrogel, and Poly N acrylamide (PNAM) hydrogel.

The present invention also relates to a hydrogel containing the EVs of the invention and possibly another biocompatible material or excipient. An alginate hydrogel is herein preferred, such as for the alginate hydrogel described in CN106538515.

In the context of the present invention, “biocompatible materials” are those classically used in biomedical applications. They are for example metals (such as stainless steel, cobalt alloys, titanium alloys), ceramics (aluminium oxide, zirconia, calcium phosphates), polymers (silicones, poly(ethylene), poly(vinyl chloride), polyurethanes, polylactides) or natural polymers (alginate, collagen, gelatin, elastin, etc.). These materials may be synthetic or natural. Biocompatible excipients are well-known in the art and do therefore not need to be detailed.

This hydrogel can be used for cosmetic or therapeutic purposes.

The present invention also concerns a pharmaceutical or a veterinary composition containing the EVs of the invention, as well as its use for treating the diseases and disorders mentioned above. It also concerns a dermatologic or cosmetic composition containing the EVs of the invention.

This pharmaceutical, veterinary or cosmetic composition may further contain other biocompatible agents (e.g., an hydrogel) as described above.

Said composition preferably contains between 1×10⁸ and 1×10¹⁴ ppEV per mL, more preferably between 1×10¹¹ and 1×10¹² ppEV per mL.

### DESCRIPTION OF THE FIGURES

FIG. 1 discloses a western blot showing the presence/absence of the CD9, CD81 and calnexin markers in the extracellular vesicles purified in the examples. Column A=cellular lysate of MDA cells/column B=cellular lysate of the MSCs of the invention in alpha-MEM/column C=EVs of the invention in alpha-MEM.

FIG. 2 discloses a Boyden chamber chemotactic migration assay showing the paracrine effect of the cells used to produce the EVs of the invention on ECFC in presence or absence of VEGF. Column T=medium control migration level in absence (−)/presence (+) of 50 ng/mL of VEGF. Column 1=cells batch 1 migration level in absence (−)/presence (+) of 50 ng/mL of VEGF. Column 2=cells batch 2 migration level in absence (−)/presence (+) of 50 ng/mL of VEGF. Column 3=cells batch 3 migration level in absence (−)/presence (+) of 50 ng/mL of VEGF.

FIG. 3 discloses the western blots showing the content of EVs derived from MSCs conditioned in three different conditions: column A=EV derived from non-stimulated MSCs, column B=EVs derived from MSCs stimulated with IL1β and IL4 (EVs of the invention), and column C=EVs derived from MSCs stimulated with LPS, as described in example 8. Six markers have been tested (CD9, CD81, Alix, Calnexin, VCAM, and CD200). 20 μg of EVs have been added per well.

FIG. 4 highlights the differential content in different angiogenesis-related proteins determined by protein array on the EVs derived from MSCs conditioned in three different conditions (non-stimulated, IL-stimulated and LPS-stimulated). The average pixel intensity is reported for each protein.

FIG. 5 discloses the western blots showing the content MSCs conditioned in three different conditions: column A=non-stimulated MSCs, column B=MSCs stimulated with IL1β and IL4 (MSCs of the invention), and column C=MSCs stimulated with LPS, as described in example 8. Six markers have been tested (CD9, CD81, Alix, Calnexin, VCAM, and CD200). 20 μg of protein lysate have been added per well.

### EXAMPLES

For simplicity and illustrative purposes, the present invention is described by referring to exemplary embodiments thereof. It will be apparent, however, to one of ordinary skill in the art that the present invention may be practiced without limitation to these specific details. In other instances, well known methods have not been described in detail so as not to unnecessarily obscure the present invention.

All the steps 1-4 below have been performed as described in the example part of PCT/EP2017/082316 which is hereby incorporated by reference.

1. Pro-Angiogenic Umbilical Cord-Derived Mesenchymal Stem Cells (MSC) Isolation

The umbilical cord has been removed from the transport solution and cut in 2-3 cm section long. Every segment containing a blood clot that cannot be removed was discarded, to avoid contamination by adherent blood cells. The sections were then disinfected by a bath of antibiotics and antifungal agents composed of αMEM+Vancomycin 1 g/L+Amoxicillin 1 g/L+Amikacin 500 mg/L+Amphotericin B 50 mg/L for 30 min at room temperature (RT). Antibiotics were extemporaneously dissolved in sterile water for injection.

The sections of umbilical cord were removed from the bath and quickly rinsed in 1×PBS at RT. The epithelial membrane was slightly sectioned without touching the vessels. The section was then detailed in slices of 0.5 cm thickness and disposed at the bottom of 150 cm² plastic flask with lid. 6 to 10 slices per flask were disposed with at least a 1 cm radius circle of free space around each slice, and left to adhere for 15 min without medium at RT.

After adhesion, complete medium (αMEM+5% Clinical Grade Platelet Lysate+2 U/mL heparin) was added carefully, to keep the explants adherent to the bottom of the flask. The flasks were then incubated at 37° C., 90% humidity and 5% CO2.

The culture medium was changed after 5 to 7 days.

At day 10 after isolation, the migration of the cells out of the explants was controlled by inverted microscopy. If a circle of adherent cells was visible around most of the explants, they were carefully removed, by picking them out of the flask, through the lid, with a sterile, disposable, single-use pair of tweezers.

From this step, the confluency of the cells was visually checked every other day and, if needed, a medium change was performed at day 17.

When the cells reached 70-90% confluency or at D20, the medium was removed, cells were washed with 30 mL of 1×PBS per flask. Cells were then removed with trypsin and collected with the old medium and centrifuged 10 min at 300 g. Supernatant was discarded and cells were then suspended in a cryopreservation solution consisting in αMEM+100 mg/mL HSA (Human Serum Albumin)+10% DMSO (DiMethyl SulfOxide) and cryopreserved.

2. MSC Cells Thawing and Culture

Cells were thawed following a classical protocol. Briefly, cryotubes were removed for liquid nitrogen and quickly plunged into a 37° C. water bath. As soon as there was no ice left in the tube, cells were diluted in preheated (37° C.) complete medium (αMEM+0.5% (v/v) ciprofloxacin+2 U/mL heparin+5% (v/v) Platelet Lysate (PL) and quickly centrifuged (300 g, RT, 5 min).

After centrifugation, cells were suspended in preheated complete medium, and assessed for number and viability (blue trypan/Malassez hemocytometer).

Cells were seeded at 4000 cells/cm² in plastic culture flask in complete medium, and incubated (90% humidity, 5% CO2, 37° C.).

3. MSC Cells Stimulation

After a few days of expansion, cells were checked for confluency. When confluency reached 30 to 50%, the old medium was discarded and replaced either by fresh complete medium for unstimulated condition, or by fresh medium completed with 10 ng/mL IL-1β and 10 ng/mL IL-4.

Cells were then incubated at least 2 days before flow cytometry experiment.

This stimulation step was instrumental in conferring a pro-angiogenic phenotype to the MSC, as described in PCT/EP2017/082316.

Several cells batches have been tested in a Boyden chamber assay for their angiogenic activity (see FIG. 2).

Briefly, ECFC (Endothelial Colony Forming Cells) migration in response to a pro-angiogenic gradient was assessed in a 24-well modified Boyden chamber, on polycarbonate membrane filter with 8 μm pore diameter (BD Biosciences) coated with 20 μg/mL fibronectin from bovine plasma (Sigma-Aldrich—F1141). Before the experiment, the bottom of a 24-well plate wells was seeded with the MSC cells at a density of 6000 cells/cm² in sextuplicate and cultivated 4 days in αMEM+1% SVF. A medium control row was realized, filled with αMEM+1% FBS.

After an overnight starvation in EBM-2 basal medium (Lonza—CC-3156) supplemented with 0.2% FBS, ECFC were loaded in starving medium into the upper part of the microchamber at 200,000 cells/well.

For each condition VEGF (Miltenyi—130-109-383) was added in three of the six wells at a concentration of 50 ng/mL as a positive control.

After 5 h of incubation, cells on the upper surface of the membrane filter were removed by wiping with a cotton swab. Then, all membranes were MGG-stained, mounted and photographed.

| | Insert 1 | Insert 2 | Insert 3 |
|---|---|---|---|
| | (n = 3) | (n = 3) | (n = 3) |
| MEM-α 1% | 95.00 | 196.67 | 259.67 |
| SVF − VEGF | | | |
| (Medium CTRL−) | | | |
| MEM-α 1% | 509.33 | 390.00 | 494.33 |
| SVF avec VEGF | | | |
| (Medium CTRL+) | | | |
| 101 − VEGF | 234.00 | 177.00 | 258.00 |
| 101 + VEGF | 439.00 | 433.67 | 573.00 |
| 201 − VEGF | 330.00 | 257.00 | 224.33 |
| 201 + VEGF | 648.33 | 537.33 | 635.33 |
| 301 − VEGF | 333.67 | 257.33 | 320.67 |
| 301 + VEGF | 564.33 | 648.00 | 599.67 |

In FIG. 2, all the (−) columns are the number of migrated ECFC in absence of VEGF, whereas all the (+) columns are the number of migrated ECFC in presence of VEGF. The control condition (T− and T+) show that the ECFC are able to enhance their migration in presence of VEGF. The tests conditions (1, 2, 3,) show that whereas batch 1 doesn't seem to affect the ECFC behavior, both batches 2 and 3 enhance the effect of VEGF on ECFC, and moreover, batch 3 even enhance ECFC migration in absence of VEGF.

As Boyden chambers prevent cell to cell contact, this effect is necessarily mediated through soluble extracellular mediators, either soluble proteins or extracellular vesicles.

4. MSC Cells Harvesting and Cryopreservation

After 2 to 3 days of expansion/stimulation, cells were checked for confluency. If confluency was up to 80%, the cells were harvested. Briefly, the old medium was discarded and cells were washed with 1×DPBS. Trypsin EDTA was added and cells were incubated 5 min at 37° C. Trypsin was neutralized with at least 2× the volume of medium, and cell suspension was harvested and assessed for number and viability.

Cells were centrifuged 10 min at 300 g. Supernatant was discarded and cells were then suspended in a cryopreservation solution consisting in αMEM+100 mg/mL HSA+10% DMSO and cryopreserved.

5. MSC Cells Thawing and Conditioned Medium Production

Cells were thawed following a classical protocol. Briefly, cryotubes or bags were removed for liquid nitrogen and quickly plunged into a 37° C. water bath. As soon as there was no ice left in the tube, cells were diluted in preheated (37° C.) αMEM and quickly centrifuged (300 g, RT, 5 min).

After centrifugation, cells were suspended in preheated complete medium, and assessed for number and viability (blue trypan/Mallassez hemocytometer).

Cells were seeded at a density of 2000 cells/cm² in the necessary number of 300 cm² plastic culture flask to produce the requested amount of conditioned medium (1T300=30 mL of conditioned medium). Cells were incubated at 90% humidity, 5% CO2, 37° C., with αMEM complemented with 0.5% (v/v) ciprofloxacin, 2 U/mL heparin and 8% (v/v) of centrifuged PL. Centrifuged PL was prepared by centrifuging PL 1 h at 6000 g and 10° C., and recovering the supernatant.

After 5 days the medium was changed. When the cells reached 80% confluency (at day 6), the medium was removed and cells were washed 3 times with PBS. 50 ml/T300 of uncomplemented αMEM was added for 24H. After 24H the medium was changed to 30 mL of either uncomplemented αMEM, or αMEM complemented with 8% of vesicles deprived PL (platelet lysate).

Cells were allowed to secrete for 36H and the medium was retrieved, centrifuged 5 min at 400 g and RT in a Heraeus Multifuge 3 S-R. The supernatant was retrieved and frozen at −80° C.

Cells were trypsinized and assayed for their number and viability.

6. Extra-Cellular Vesicles Isolation and Characterization

Extra-cellular vesicles derived from such MSC can be isolated by any methods known in the art, such as, but not limited to, ultracentrifugation, ultrafiltration, density gradient, size-exclusion chromatography, kit-based precipitation, immune-affinity capture, microfluidic devices.

In this experiment, the fraction enriched in extra-cellular vesicles was separated by ultrafiltration of the conditioned medium followed by Size Exclusion Liquid Chromatography (SEC).

The analysis of number and size distribution of extra-cellular vesicles was performed using Nanoparticle Tracking Analysis (Nanosight).

Also, the content of the EVs of the invention has been assessed by Western Blot, using the following conditions.

Material and Reagents:

Lysis buffer (RIPA):

| Sodium deoxycholate | 1% | |
|---|---|---|
| SDS | 0.1% | |
| Tris. Cl pH 7.4 | 20 | mM |
| EDTA | 1 | mM |
| NaCl | 150 | mM |

| | |
|---|---|
| Triton x100 | 1% |

Protease Inhibitor Cocktail (CIP 100X), Sigma ref. P8340

Cells Lysis:

100 μl of cold RIPA containing CIP at 1× final concentration has been added to 1×10⁶ MSC cells.

The resulting mixture has been incubated for 10 mn on ice, centrifuged for 15 mn at 4° C. for 20 min and then the supernatant has been recovered.

The dosage of proteins has been done by using the micro BCA Protein Assay Kit Pierce Ref 23235.

The electrophoresis was done on Novex Nosex 4-12% Bis-Tris Protein Gels 1.5 mm, 10 wells (Life Technologies, NP0335PBOX) or NuPAGE® MOPS SDS Running Buffer (20×) (Life Technologies, NP0001). The samples have been denatured for 10 mn at 70° C. with ¼ volume of LDS buffer (Invitrogen NP0007, 4×) with or without DTT (500 mM). The transfer was done with Mini Trans-Blot Electrophoretic cell membrane transfer, ref: 170-3930 on a membrane of PVDF Amersham, ref: RPN303LFP activated with absolute ethanol and rinsed.

The following Materials were used for revealing the proteins:

TBS10X, BioRad ref. 1706435

Blocking buffer (TBS milk): TBS 1X_Tween 0.1% skimmed milk 5%

Washing Buffer (TBS): TBS 1X_Tween 0.1%

AC Buffer (TBS_AC): TBS 1X_Tween 0.1% _Milk 0.3%

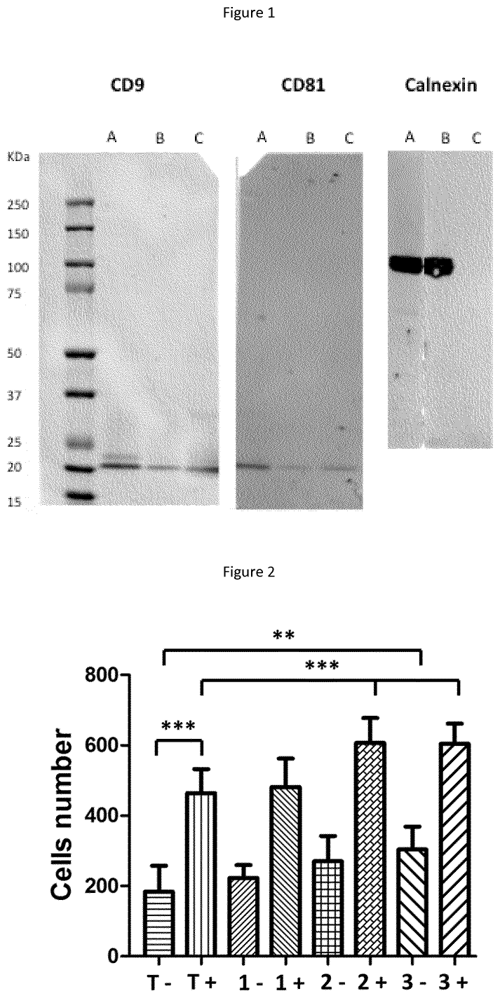

The presence of membrane proteins CD9 (Biolegend—312102) and CD81 (Biolegend—349501) was analyzed in Western Blot (FIG. 1). Both the markers CD9 and CD81 were present in the fraction of the purified EVs (column C) along with the MDA cells lysate (column A—positive control) and the source cell lysate (column B). The reticulum marker Calnexin (Elabscience—E-AB-30723) was not, showing that the fraction was correctly purified.

Fluorimetry has been performed by using Alexa Fluor 680 antibody, Thermofisher GAM (ref: A21058) or GAR (ref: A21076) at 1/10000^{th}.

Chemiluminescence has been performed by using HRP Antibody, Bio Rad GAM (ref: 170-6516) or GAR (ref: 170-6515) at 1/5000th.

7. Comparison of EV Derived from UCMSCs Stimulated by LPS and EV Derived from UCMSCs Stimulated by the Combination of IL1β and IL4 (EVs of the Invention).

Dongdong Ti & al (Journal of translational medicine, 2015) describes extra-cellular vesicles purified from umbilical cord MSCs that are pre-conditioned with the pro-inflammatory factor LPS.

Yunbing Wu & al (BioMed Research International, 2017) describes anti-inflammatory properties of extra-cellular vesicles derived from unstimulated mesenchymal stem cells obtained from human umbilical cord.

None of these publications proposes to add IL1β and/or IL4 for stimulating the MSCs from which the EVs of the invention are derived.

The purpose of the present example is to demonstrate that EV derived from the CD106^{high} CD151⁺Nestin⁺ MSCs of the invention are distinct from those described in the prior art, because they are secreted by cells that have been cultivated in a particular conditioning medium containing pro-inflammatory growth factors such as IL1β and IL4.

A comparison of the EV of the invention with the EV derived from unstimulated MSCs, or from MSCs stimulated by LPS demonstrated that the EVs of the invention exhibit molecular characteristics that distinguish they indeed from the EVs of the prior art, e.g., in terms of protein content or surface markers.

7.1. Production of Conditioned Media (Culture Supernatants) of Umbilical Cord-Derived MSCs Cultured in 3 Different Conditioning Media:

7.1.1. Preparation of the 3 Conditioning Media

Three conditioning media were prepared as follows:

NS=Control medium (without conditioning pro-inflammatory factor):

For a 675 mL αMEM bag (final concentration: 10 ng/mL)
1—Put an injection site in one of the outlet port.
2— Inject with a needle 290 μL of heparin 5000 U/mL (final concentration 2 U/mL). Ensure that no heparin is left in the injection site by suction/discharge.
3—Put an injection site in the outlet port of a bag of Clinical Platelet Lysate (CPL) and take 56.8 mL of centrifuged CPL with a 50 mL LL syringe.

S1=IL1β—IL4 conditioning medium (“second medium” of the method as described in WO 2018/108859):

For a 675 mL αMEM bag (final total interleukine concentration: 10 ng/mL)
4—Put an injection site in one of the outlet port.
5— Inject with a needle 290 μL of heparin 5000 U/mL (final concentration 2 U/mL). Ensure that no heparin is left in the injection site by suction/discharge.
6—Put an injection site in the outlet port of a bag of CPL and take 56.8 mL of centrifuged CPL with a 50 mL LL syringe.
7—Inject the CPL in the αMEM bag and homogenise with the syringe.
8—Cytokine addition:
   1. Thaw a 73.2 μL aliquot of each interleukine (IL) (C=100 μg/mL).
   2. Use a 1 mL needled syringe to sample 400 μL of complete medium, and mix it with the ILs.
   3. Take the medium containing the ILs and add it to the bag of complete medium.

LPS Conditioning Medium:

For a 675 mL αMEM bag (final LPS concentration: 100 ng/mL)
1—Put an injection site in one of the outlet port.
2—Inject with a needle 290 μL of heparin 5000 U/mL (final concentration 2 U/mL). Ensure that no heparin is left in the injection site by suction/discharge.
3—Put an injection site in the outlet port of a bag of CPL and take 56.8 mL of centrifuged CPL with a 50 mL LL syringe.
4— Inject the CPL in the αMEM bag and homogenise with the syringe.
5—LPS addition:
   1. Thaw a 73.2 μL aliquot of LPS (C=1 mg/mL).
   2. Use a 1 mL needled syringe to sample 400 μL of complete medium, and mix it with the LPS.
   3. Take the medium containing the LPS and add it to the bag of complete medium.

Centrifuged Clinical Platelet Lysate (CPL) Preparation:

Centrifuged CPL was used for the stimulation step, in order to start “cleaning” the cells from exogenous EV derived from the CPL.

1—Put an injection site in the outlet port.

2—Transfer the CPL in 50 mL plastic tubes.

3—Centrifuged at 6000 g and 4° C. for 1 hour.

4—Transfer the supernatant in a new container.

7.1.2. Thawing and Amplification:

Umbilical cord-derived mesenchymal stem cells, stored in gaseous nitrogen after isolation and passage 1, have been thawed following a classical protocol. Briefly, the bag was removed from storage and quickly plunged into a 37° C. water bath. As soon as there was no ice left in the bag, cells were diluted in preheated (37° C.) complete medium (αMEM+0.5% (v/v) ciprofloxacin+2 U/mL heparin+5% (v/v) PL) and quickly centrifuged (300 g, RT, 5 min).

After centrifugation, the cells were suspended in preheated complete medium, and assayed for cell count and viability (blue trypan/Malassez hemocytometer).

The cells were seeded at 2000 cells/cm² in two cell stacks 1 (CS1) in complete medium, and incubated (90% humidity, 5% CO2, 37° C.) for seven days.

After seven days, the CS1 were rinsed with 100 mL of PBS, and 25 mL of Trypzean per CS1 were added to harvest the cells. After 10 min in the incubator the Trypzean was neutralized with a total of 100 mL of complete medium (50 mL per CS1). Cells were pooled and assessed for number and viability.

7.1.3. Stimulation:

T300 were seeded at 6000 cells/cm².

After one day, the medium was changed for the appropriate conditioning medium. The cells were stimulated for 2 days without medium change.

7.1.4. Starving and EV Secretion:

After the stimulation the medium was discarded and the cells were rinsed three times with PBS. Cells were then starved 24H with αMEM+/−a pro-inflammatory factor (combination of 10 ng/mL of IL1β and of IL4 or 100 ng/mL of LPS).

After the starving period, the cells were again rinsed three times with PBS then each flask was loaded with 30 mL of αMEM+/−a pro-inflammatory factor (IL or LPS) for 72 hours.

The supernatant was collected in 50 mL plastic tubes and centrifuged for 5 min at 400 g at room temperature. The supernatant of each condition was pooled in a 500 mL bottle and a small aliquot of 1 ml was separately frozen at −80° C. along the bottles.

For each condition, 3 T300 flasks were trypsinized to evaluate the number of cells, and cells were cryopreserved in cryotubes.

Cell Culture and Conditioning Reagents:

| Manufacturer | Reagent | Reference | Batch number |
|---|---|---|---|
| Macopharma | αMEM | BC0110020 | 112611484DM |
| Macopharma | Clinical Platelet | Multi PL100i | 11217335DM |
| | Lysate (virally | | |
| | inactivated) | | |
| Sigma | Trypzean | T3449-500ML | RNBG4130 and |
| Sanofi | Heparin | CIP | 70579 |
| | CHOAY 5000 | 3400955243861 | |
| | UI/mL | | |
| Macopharma | PBS | BC0120030 | 11233414DM |
| CellGenix | IL-1β | 201-GMP-01M | 1011QB11a |
| CellGenix | IL-4 | 204-GMP-01M | 1003PC22b |
| Macopharma | CS1 | Cellstack 1 | 29815014 |
| Dutscher | 300 cm² culture | 190301 | 20180414 |
| | flasks | | |
| Pharma | Ciprofloxacine | Cipro | 16102089 |
| Sigma | LPS | L6529-1MG | 028M4138V |

7.2. Phenotypic Characterisation of the Cells Contained in the 3 Conditioned Media

A phenotypic characterization of the cells cultured using the 3 conditioning media was performed by cytometry analysis to assess the efficiency of the stimulation.

As expected, the cells conditioned in the media S1 and LPS exhibited a different phenotype, the pro-angiogenic marker CD106 being expressed at higher levels in the conditioned media S1 (stimulation of the cells by ILs as described in WO 2018/108859).

| | | S1 = IL-Stimulated | |
|---|---|---|---|
| | | as described in WO | |
| | Unstimulated | 2018/108859 | LPS-Stimulated |
| Cell number | 808 000 cells | 1 693 000 cells | 1 666 000 cells |
| Viability | 91% | 95% | 98% |
| CD90+ | 99.96% | 99.38% | 99.99% |
| CD106+ | 12.21% | 89.93% | 23.14% |
| HLA-DR+ | 0.87% | 1.88% | 0.33% |
| CD31+ | 7.28% | 1.11% | 0.25% |
| CD34+ | 7.56% | 0.62% | 0.56% |
| CD73+ | 99.94% | 99.94% | 99.99% |
| CD45+ | 4.66% | 1.34% | 0.82% |
| CD151+ | 97.39% | 99.25% | 100.00% |
| CD105+ | 99.59% | 99.70% | 99.94% |

Cytometry Reagents Used for Cytometry Analysis:

| Manufacturer | Reagent | Reference | Batch number |
|---|---|---|---|
| Dutscher | 1X DPBS | P04-36500 | 9110816 |
| R&D System | Human polyvalent IgG | 1-001-A | |
| | (2 mg/mL in PBS) | | |
| LFB | HSA 20% | Vialebex 20% | |
| Sigma | 37% filtered Formalin | F1635 | |
| Miltenyi | MACS Comp Bead | 130-097-900 | 5180522233 |
| | Kit anti-Mouse Igk | | |
| Dutscher | 96-wells conic bottom | 651101 | |
| | plate | | |
| BD Falcon | Cytometry tubes | 352053 | |
| Miltenyi | CD34-PE, human | 130-081-002 | 5180511513 |
| Beckman | CD31-FITC, human | IM143U | 33 |
| Coulter | | | |
| Beckman | CD90-FITC, human | IM1839 | 35 |
| Coulter | | | |
| Miltenyi | Anti-HLA-DR-FITC, | 130-111-788 | 5171121099 |
| | human | | |
| Miltenyi | CD105-PE, human | 130-094-941 | 5170105175 |
| Miltenyi | CD45-FITC, human | 130-110-631 | 5180209339 |
| Miltenyi | Mouse IgG2a-PE | 130-091-835 | 5160804636 |
| Miltenyi | Mouse IgG1-FITC | 130-092-213 | 5160804642 |
| Miltenyi | REA Control (S)-FITC | 130-104-610 | 5161209090 |
| Miltenyi | MACS Comp Bead | 130-104-693 | 5160901338 |
| | Kit anti-Mouse REA | | |
| BD | PE Mouse IgG1, □ | 555749 | 6070641 |
| Biosciences | Isotype Control | | |
| BD | PE Mouse | 555647 | 8025927 |
| Biosciences | Anti-Human CD106 | | |
| | 51-10C9 RUO | | |
| BD | PE Mouse | 550257 | 6237614 |
| Biosciences | Anti-Human CD73 | | |
| | AD2 RUO | | |
| BD | PE Mouse | 556057 | 5134578 |
| Biosciences | Anti-Human CD151 | | |
| | 14A2.H1 RUO | | |

7.3. EV Purification

In this experiment, the fractions enriched in extra-cellular vesicles were separated by ultrafiltration of the 3 conditioned media.

The analysis of the number and size distribution of extra-cellular vesicles was performed using Nanoparticle Tracking Analysis (Nanosight 300 from Malvern-Panalytical).

The results of EV purification are presented below:

Yield:

| | NS | S1 | LPS |
|---|---|---|---|
| Concentration (vesicles/mL) | 2.90E+09 | 8.65E+08 | 2.81E+09 |
| Initial Volume in | 525 | 538 | 539 |
| conditioned medium or | | | |
| MC 0.45μ | | | |
| Conc° EV after tangential | 5.96E+10 | 1.43E+11 | 8.54E+10 |
| ultrafiltration (particules/mL) | | | |
| Number of frozen tubes (/0.5 mL) | 12 | 12 | 11 |
| Number of EV/tube | 2.98E+10 | 7.15E+10 | 4.27E+10 |
| Total number of EV | 3.58E+11 | 8.58E+11 | 4.70E+11 |
| Total Protein Concentration (mg/mL) | 2.0 | 2.3 | 2.4 |

Size:

| | Conditioned Medium | | | | | | | | | | | |
|---|---|---|---|---|---|---|---|---|---|---|---|---|
| | (MC) 0.45μ | | | | | | EV | | | | | |
| | NS | SD | S1 | SD | LPS | SD | NS | SD | S1 | SD | LPS | SD |
| mode | 145.1 | 6.3 | 136.7 | 7.2 | 138.1 | 7.2 | 134.6 | 3.1 | 140.5 | 5.5 | 131.4 | 4.2 |
| D10 | 127.2 | 5.4 | 111.7 | 2 | 113 | 1.2 | 121.1 | 2.8 | 123.1 | 1.5 | 119.4 | 1.1 |
| D50 | 222.6 | 6.9 | 147.3 | 4.1 | 146.8 | 2.2 | 159.7 | 5.3 | 159.8 | 1.6 | 153.3 | 4.3 |
| D90 | 439 | 16.4 | 209.5 | 11.7 | 242 | 4.1 | 277.5 | 10.8 | 269.4 | 6.1 | 278.3 | 7 |

Yield/Cells:

| | | | | | | Yield |
|---|---|---|---|---|---|---|
| | | | EV in the | | | EV/cells/hour |
| | Cells | | conditioned | EV after | Yield | of secretion |
| Condition | harvested | Viability | medium | purification | EV/cells | (72 h) |
| Unstimulated | 1.10E+08 | 95.81% | 1.52E+12 | 3.58E+11 | 3251 | 45 |
| S1-Stimulated | 1.02E+08 | 90.68% | 4.65E+11 | 8.58E+11 | 8412 | 117 |
| LPS-stimulated | 1.04E+08 | 91.75% | 1.51E+12 | 4.70E+11 | 4526 | 63 |

7.3. Characterisation of the EVs Obtained from NS/S1/LPS-Conditioned MSCs

7.3.1. EV and MSC Protein Content Analysis by Western Blot

The content of the EVs and the MSCs obtained in the 3 conditions (NS/S1/LPS) has been assessed by Western Blot using the following conditions.

Material and Reagents:

Lysis buffer (RIPA):
Sodium deoxycholate 1%
SDS 0.1%
Tris.HCl pH 7.4 20 mM
EDTA 1 mM
NaCl 150 mM
NP40: 1%

Protease Inhibitor Cocktail (CIP 100×), Sigma ref. P8340

Cell Lysis (for the MSCs):

Add 100 μl/1×10⁶ MSC cells of cold RIPA containing CIP at 1× final concentration.

Incubate for 10 mn in ice, centrifuge at 4° C. for 20 min at 10000 g and then recover the supernatant.

The EV containing fraction was solubilized following addition of an equal volume of ice-cold 2×RIPA lysis buffer.

The dosage of proteins has been done by using the micro BCA Protein Assay Kit Pierce Ref 23235.

The electrophoresis was done on Novex Nosex 4-12% Bis-Tris Protein Gels 1.5 mm, 10 wells (Life Technologies, NP0335PBOX) or NuPAGE® MOPS SDS Running Buffer (20×) (Life Technologies, NP0001). The samples have been denatured for 10 mn at 70° C. with ¼ volume of LDS sample buffer (Invitrogen NP0007, 4×) with or without DTT (500 mM). The transfer was done with Mini Trans-Blot Electrophoretic cell membrane transfer, ref: 170-3930 on a membrane of PVDF Amersham, ref: RPN303LFP activated with absolute ethanol and rinsed.

The following Materials were used for revealing the proteins:

TBS10X, BioRad ref. 1706435

Blocking buffer (TBS milk): TBS 1X_Tween 0.1% skimmed milk 5%

Washing Buffer (TBS): TBS 1X_Tween 0.1%

AC Buffer (TBS_AC): TBS 1X_Tween 0.1% _Milk 0.3%

Fluorimetry has been performed by using Alexa Fluor 680 antibody, Thermofisher GAM (ref: A21058) or GAR (ref: A21076) at 1/10000^{th}.

Chemiluminescence has been performed by using HRP Antibody, Bio Rad GAM (ref: 170-6516) or GAR (ref: 170-6515) at 1/5000th.

Antibodies anti-CD9: Biolegend—312102

Antibodies anti-CD81: Biolegend—349501

Antibodies anti-Calnexin: Elabscience—E-AB-30723

Antibodies anti-VCAM: Bio-Rad VMA00461

Antibodies anti-CD200: Bio-Techne 2AF2724

Results:

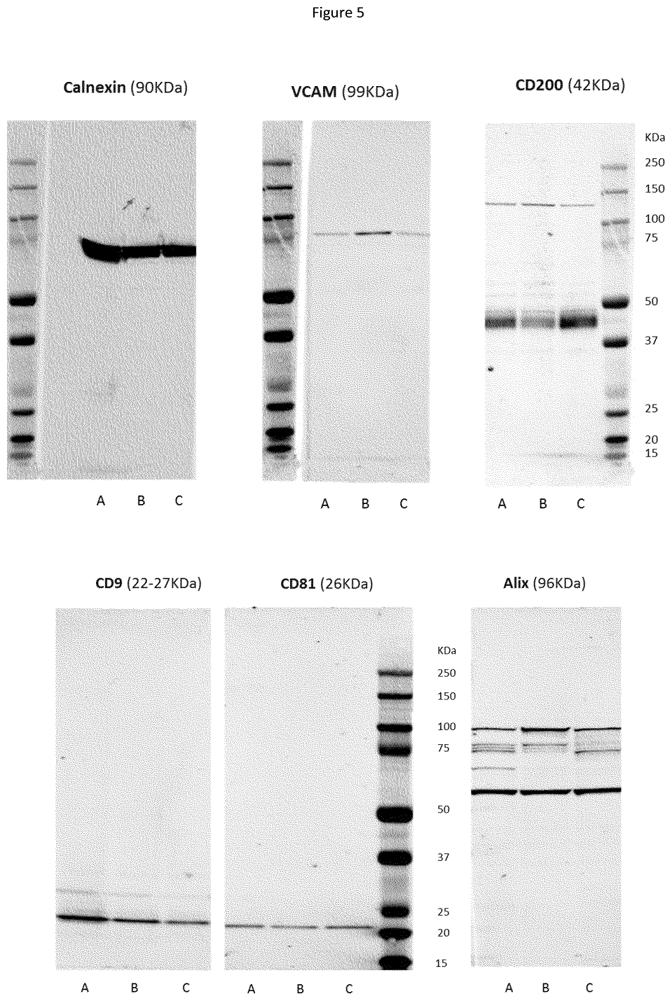

As expected, the pro-angiogenic marker CD106/VCAM was detectable in column B (MSCs of the invention) and completely absent in column C (MSCs in LPS condition), and in column A (negative control), as expected. The membrane glycoprotein CD200, a second pro-angiogenic marker, was present in the MSCs of the S1 fraction and not in the MSCs in the LPS fraction, nor in the column A (negative control) (see FIG. 5).

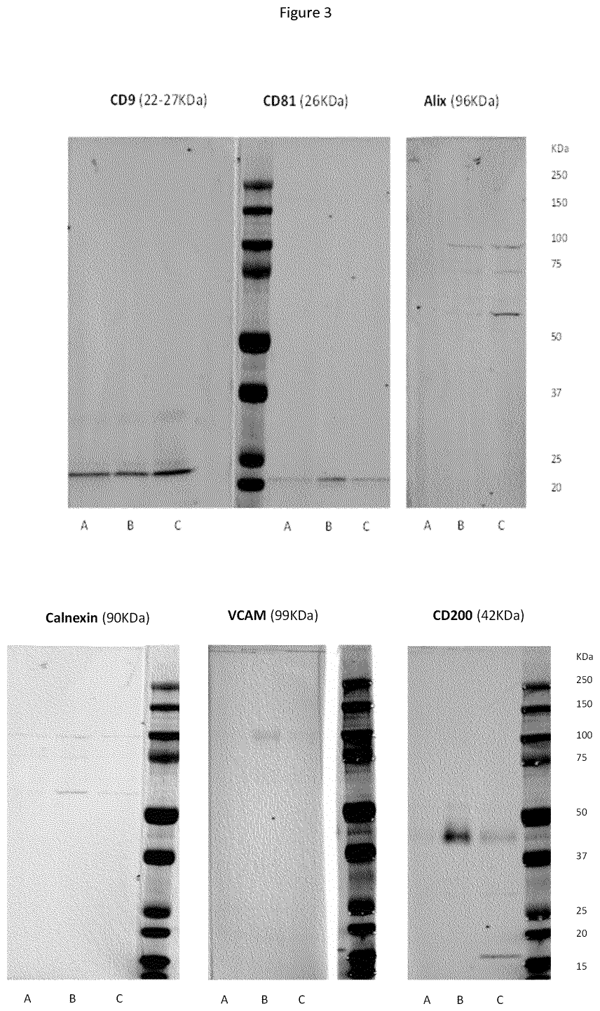

In the purified EVs (FIG. 3), both CD9 and CD81 membrane proteins were present in all the fractions (columns A, B and C) along with the MDA cells lysate (positive control) (FIG. 3).

The reticulum marker Calnexin was not detected, showing that the EV fraction was correctly purified.

The pro-angiogenic marker CD106/VCAM was detectable in column B (EVs of the invention) and completely absent in column C (EVs in LPS condition), and in column A (negative control), showing that the membrane markers of the MSCs of the invention are transferred to the EVs (FIG. 3).

The membrane glycoprotein CD200, a second pro-angiogenic marker, was present in the EVs of the S1 fraction and not in the EVs of the LPS fraction, nor in the column A (negative control)(FIG. 3).

7.3.2 Angiogenesis Protein Array

The EVs derived from the MSCs cultivated in NS, S1 and LPS conditioned media were compared with an angiogenesis proteome array (R&D Systems—ARY007).

The lysis Buffer was prepared as follows:
1. For 50 mL: Dissolve 157.6 mg of Tris-HCL in 10 mL of water, 400.3 mg of NaCl in 10 mL of water and 37.2 mg of EDTA in 10 mL of water.
2. Adjust to pH=8.
3. Add 0.5 mL of triton X-100, 5 mL of Glycerol, 50 μL of Aprotinin 10 mg/mL, 50 μL of Leupeptin 10 mg/mL and 500 μL of Pepstatin 1 mg/mL.
4. QSP 50 mL of sterile water.

The EVs derived from the MSCs obtained in the NS, S1 and LPS media were solubilized in the lysis buffer:
NS: 150 μL of EVs were suspended in 1 mL of Lysis Buffer
S1: 130.4 μL of EV were suspended in 1 mL of Lysis Buffer
LPS: 125 μL of EV were suspended in 1 mL of Lysis Buffer

A total amount of proteins in the lysates of 300 μg was quantified with a BCA assay, thereby confirming the efficiency of the lysis.

The EVs lysates were then pipeted up and down for resuspension and gently rocked at 2-8° C. for 30 minutes, then micro-centrifuged at 14,000×g for 5 minutes. The supernatant was transferred into a clean test tube and assayed following the manufacturer's instructions.

Angiogenesis Proteome Array Reagents:

| Manufacturer | Reagent | Reference | Batch number |
|---|---|---|---|
| R&D | Angiogenesis proteome | ARY007 | |
| Systems | array | | |
| Merk | Tris-HCL | 1547-0100 | |
| Sigma | NaCl | S3014 | |
| Biosolve | EDTA | 05142357 | |
| Prolabo | Triton X-100 | 81338 | |
| Invitrogen | Glycerol | 15514-011 | |
| Tocris | Aprotinin | 4139 | 3 |
| Tocris | Leupeptin | 1167 | 20 |
| Tocris | Pepstatin A | 1190 | 20 |
| Versylene | Eau Sterile | B230521 | 13MFP291 |
| Fresnuis | | | |
| Thermo | Micro BCA ™ | 23252 | MC154396 |
| Scientific | Protein Assay Kit | | |

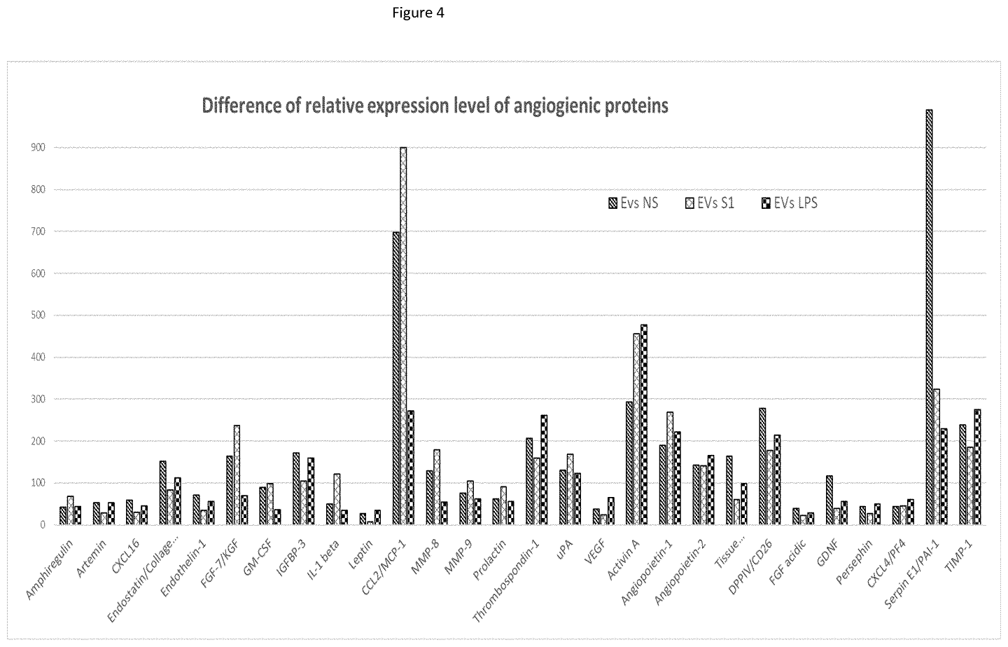

The graph on FIG. 4 presents the results for proteins which were significantly differentially expressed in the 3 samples. 28 pro-angiogenic proteins out of the 59 analyzed in the array presented differences of expression in the S1 and LPS samples, confirming that the EVs of the invention exhibit characteristics different from the EVs of the prior art, notably in terms pro-angiogenic protein content.

### BIBLIOGRAPHIC REFERENCES

Shabbir, A.; Cox, A.; Rodriguez-Menocal, L.; Salgado, M.; Badiavas, E. V. Mesenchymal stem cell exosomes induce proliferation and migration of normal and chronic wound fibroblasts, and enhance angiogenesis in vitro. Stem Cells Dev. 2015, 24, 1635-1647]
Uyen Thi Trang Than, Dominic Guanzon, David Leavesley, and Tony Parker—Association of Extracellular Membrane Vesicles with Cutaneous Wound Healing—Int. John Mol Sci 2017, 18, 956
Graça Raposo, Hans W. Nijman, Willem Stoorvogel, Richtje Leijendekker, Clifford V. Harding, Cornelis J. M. Melief, and Hans J. Geuze—B Lymphocytes Secrete Antigen-presenting Vesicles—J. Exp. Med. Volume 183 March 1996 1161-1172
Bernd Giebel, Lambros Kordelas, Verena Börger—Clinical potential of mesenchymal stem/stromal cell-derived extracellular vesicles—Stem Cell Investig 2017; 4:84
Donald. G. Phinney, Mark Pittenger—Concise Review: MSC-Derived Exosomes for Cell-Free Therapy—Stem Cells 2017; 35:851-858
Liang, X.; Zhang, L.; Wang, S.; Han, Q.; Zhao, R. C. Exosomes secreted by mesenchymal stem cells promote endothelial cell angiogenesis by transferring miR-125a. J. Cell Sci. 2016, 129, 2182-2189.
Zhang, B.; Wu, X.; Zhang, X.; Sun, Y.; Yan, Y.; Shi, H.; Zhu, Y.; Wu, L.; Pan, Z.; Zhu, W. Human umbilical cord mesenchymal stem cell exosomes enhance angiogenesis through the WNT4/_-catenin pathway. Stem Cells Trans. Med. 2015, 4, 513-522.
Uyen Thi Trang Than, Dominic Guanzon, David Leavesley and Tony Parker—Association of Extracellular Membrane Vesicles with Cutaneous Wound Healing—Int. J. Mol. Sci. 2017
Sabrina Roya, Fred H. Hochberg b and Pamela S. Jonesc—Extracellular vesicles: the growth as diagnostics and therapeutics; a survey—J. of Extracellular Vesicles, 2018
Anna Otte, Vesna Bucan, Kerstin Reimers, and Ralf Hass. Mesenchymal Stem Cells Maintain

Long-Term In Vitro Stemness During Explant Culture. Tissue Engineering Part C: Methods. November 2013, 19(12): 937-948
Van Pham, P., Truong, N. C., Le, P. T B. et al. Isolation and proliferation of umbilical cord tissue derived mesenchymal stem cells for clinical applications. Cell Tissue Bank 2016 17: 289
Konoshenko M Y, Lekchnov E A, Vlassov A V, Laktionov P P. Isolation of Extracellular Vesicles: General Methodologies and Latest Trends. Biomed Res Int. 2018
Ruenn Chai Lai, Fatih Arslan May May Lee, Newman Siu Kwan Sze, Andre Choo, Tian Sheng Chen, Manuel Salto-Tellez, Leo Timmers-Chuen Neng Lee, Reida Menshawe El Oakley, Gerard Pasterkamp, Dominique P. V. de Kleijn, Sai Kiang Lia, —Exosome secreted by MSC reduces myocardial ischemia/reperfusion injury—Stem Cell Research 2010 4, 214-222
Han Z. C., et al, New insights into the heterogeneity and functional diversity of human mesenchymal stem cells. Bio-medical Materials and Engineering 28 (2017) S29-S45
Du W. et al, VCAM-1+ placenta chorionic villi-derived mesenchymal stem cells display potent pro-angiogenic activity. Stem Cell research & therapy (2016) 7:49
Dongdong Ti et al. LPS-preconditioned mesenchymal stromal cells modify macrophage polarization for resolution of chronic inflammation via exosome-shuttled let-7b, Journal of translational medicine, 2015, vol. 13, n° 1
Yunbing Wu et al. Exosomes derived from Human Umbilical Cord Mesenchymal Stem Cells relieve Inflammatory Bowel Disease in Mice. BioMed Research International, 2017, pages 53566760-12
Katoh M. & Katoh M. CD157 and CD200 at the crossroads of endothelial remodeling and immune regulation. Stem Cell Investig. 2019; 6: 10

1. A composition comprising extracellular vesicles (EV) obtained by:
(i) culturing mesenchymal stem cells obtained from a biological tissue or fluid in a first culture medium deprived of growth factors, so as to generate a population of cultured undifferentiated mesenchymal stem cells,
(ii) contacting said population of cultured undifferentiated mesenchymal stem cells with a second culture medium containing at least two pro-inflammatory growth factors, thereby generating CD106^{high} CD151⁺Nestin⁺ mesenchymal stem cells,
(iii) Culturing said MSCs in a EV-free culture medium, under conditions permitting their expansion;
and
iv) Purifying the EVs from the cells obtained in step (iii).

2. The composition of claim 1, wherein said second culture medium used in step (ii) contains at least two pro-inflammatory growth factors chosen among TNFα, IL1, IL4, IL12, IL18, and IFNγ, preferably chosen from IL1, IL4, IL12, and IL18.

3. The composition of claim 1, wherein said EV-free culture medium is supplemented with a mixture of IL1 and IL4, preferably of IL1β and IL4.

4. The composition of any one of claims 1 to 3, wherein said EVs are obtained from undifferentiated MSCs derived from a biological tissue selected from placenta, umbilical cord or placental membranes (chorion, amnion) or their components, or from a biological fluid such as umbilical cord blood, placenta blood or amniotic fluid.

5. The composition of any one of claims 1 to 4, wherein said EVs are obtained from undifferentiated MSCs derived from umbilical cord, preferably by cell isolation from an explant of umbilical cord fragment.

6. The composition of any one of claims 1 to 4, wherein said EVs are obtained from undifferentiated MSCs derived from placenta, preferably by cell isolation from a placenta tissue fragment.

7. The composition as defined in any one of claims 1 to 6, for use for treating subjects suffering from an ischemic disease, from a disorder of the circulatory system, from an immune disease, from an organ injury or disorder or from an organ function failure.

8. The composition for use according to claim 7, wherein said disease or disorder is chosen in the group consisting of: type-1 diabetes mellitus, type-II diabetes, GVHD, aplastic anemia, multiple sclerosis, Duchenne muscular dystrophy, rheumatoid arthritis, cerebral stroke, idiopathic pulmonary fibrosis, dilated cardiomyopathy, osteoarthritis, cirrhosis, liver failure, kidney failure, peripheral arterial occlusive disease, critical limb ischemia, peripheral vascular disease, heart failure, diabetic ulcer, fibrotic disorders, synechia and endometrial disorders.

9. The composition for use according to claim 7, wherein said disease or disorder is a skin or a mucous membrane disease or disorder, preferably a diabetic ulcer, an ulcer, a trauma, a burn, a scald, a wound or wound healing problem, a Decubitus ulcer, a wart, a synechia, an endometrial disorder or a fibrotic disorder of the gastro-intestinal tract such as anal fistula.

10. A pharmaceutical, veterinary, a diagnostic or a cosmetic composition containing the EVs as defined in any one of claims 1 to 6.

11. The pharmaceutical or veterinary composition of claim 10, further containing an hydrogel or other bio-compatible material.

12. A topical formulation containing the EVs as defined in any one of claims 1 to 6.

13. The pharmaceutical composition of claim 10 or 11 or the topical formulation of claim 12, for use for treating subjects suffering from an ischemic disease, from a disorder of the circulatory system, from an immune disease, from a fibrotic disorder, from an organ injury or from an organ function failure.

14. The pharmaceutical composition of claim 10 or 11 or the topical formulation of claim 12, for use for treating subjects suffering from a disease or disorder chosen in the group consisting of: type-1 diabetes mellitus, type-II diabetes, GVHD, aplastic anemia, multiple sclerosis, Duchenne muscular dystrophy, rheumatoid arthritis, cerebral stroke, idiopathic pulmonary fibrosis, dilated cardiomyopathy, osteoarthritis, cirrhosis, liver failure, kidney failure, peripheral arterial occlusive disease, critical limb ischemia, peripheral vascular disease, heart failure, diabetic ulcer, fibrotic disorders, synechia and endometrial disorders.

15. A dermatologic or a cosmetic composition containing the EVs as defined in any one of claims 1 to 6.

16. Use of the dermatologic or cosmetic composition as defined in claim 15 for regenerating the skin or mucosal cells, improving the skin or mucosal membrane aspect or for correcting a skin or mucosal membrane defect, such as dark patches, spots, acne, wrinkles, dryness.

17. Use of the dermatologic or cosmetic composition as defined in claim 15 for healing a skin injury or disorder, such as a burn, a scar, an angioma, a mole, or a wart.

18. A medical device containing the EV as defined in any one of claims 1 to 6.

19. The medical device of claim 18, wherein it is a bandage, a patch, a stent, an endoscope, or a syringe.

20. A delivery system containing the EV as defined in any one of claims 1-6.

21. The delivery system of claim 20, wherein it is micro or nano-vesicles of biopolymers, lipids or nanoparticles.

22. A method to prepare extracellular vesicles (EV), said method comprising the steps of:
(i) culturing mesenchymal stem cells obtained from a biological tissue or fluid in a first culture medium deprived of growth factors, so as to generate a population of cultured undifferentiated mesenchymal stem cells,
(ii) contacting said population of cultured undifferentiated mesenchymal stem cells with a second culture medium containing at least two pro-inflammatory growth factors, thereby generating CD106^{high} CD151⁺Nestin⁺ mesenchymal stem cells,
(iii) Culturing said MSCs in a EV-free culture medium, under conditions permitting their expansion;
and
iv) Purifying the EVs from the cells obtained in step (iii).

23. The method of claim 22, wherein said pro-inflammatory growth factors are chosen among TNFα, IL1, IL4, IL12, IL18, and IFNγ, preferably chosen from IL1, IL4, IL12, and IL18.

24. The method of claim 22 or 23, wherein said pro-inflammatory growth factor is a mixture of IL1 and IL4, preferably of IL1β and IL4.

25. The method of any one of claims 22 to 24, wherein said undifferentiated MSCs are derived from a biological tissue selected from placenta, umbilical cord or placental membranes (chorion, amnion) or their components, or from a biological fluid such as umbilical cord blood, placenta blood or amniotic fluid.

26. The method of any one of claims 22 to 25, wherein said undifferentiated MSCs are obtained by cell isolation from explant tissues.

27. The method of any one of claims 22 to 26, wherein said undifferentiated MSCs are derived from umbilical cord, preferably by cell isolation from an explant of umbilical cord fragment.

28. The method of any one of claims 22 to 26, wherein said undifferentiated MSCs are derived from placenta, preferably by cell isolation from a placenta tissue fragment.

29. The method of any one of claims 22 to 28, wherein said second culture medium contains between 1 and 100 ng/ml of Interleukine 1, preferably of Interleukine 1β.

30. The method of any one of claims 22 to 29, wherein said second culture medium contains between 1 and 100 ng/ml of Interleukine 4.

31. The method of any one of claims 22 to 30, wherein said EV-free culture medium is supplemented with a mixture of IL1 and IL4, preferably of IL1β and IL4.

32. The method of any one of claims 22 to 31, wherein the EVs are purified by ultrafiltration.
